(19) United States
(12) Patent Application Publication
(10) Pub. No.: US 2024/0390234 A1
(43) Pub. Date: Nov. 28, 2024

(54) Enteral Feeding System

(71) Applicant: Avent, Inc., Alpharetta, GA (US)

(72) Inventors: James M. Takeuchi, Woodstock, GA (US); Sarah M. Sarkissian, Roswell, GA (US); Jeffrey J. Farmer, Roswell, GA (US)

(21) Appl. No.: 18/322,656

(22) Filed: May 24, 2023

(51) Int. Cl. A61J 15/00

(52) U.S. Cl. CPC A61J 15/0076

(57) ABSTRACT

An enteral feeding system includes a nutrition adapter, a first tube, a cartridge, a second tube, and a feeding tube adapter formed together as one piece. The nutrition adapter includes a coupling configured to couple to a source of enteral nutrition. The first tube has a proximal end that is coupled to the coupling of the nutrition adapter. The cartridge is configured for insertion into a peristaltic pump and includes a cartridge tube defining a cartridge inlet outlet coupled to a distal end of the first tube and a cartridge outlet. The second tube has a proximal end coupled to the cartridge outlet. The feeding tube adapter is coupled to a distal end of the second tube and is configured to be coupled to a feeding tube receiving port such that a lumen of the feeding tube adapter is coupled to a lumen of the second tube.

### FIELD OF THE INVENTION

The subject matter of the present invention relates generally to a custom combination giving set/extension set for administration of enteral nutrition using a peristaltic pump.

### BACKGROUND

There are many issues encountered by enteral feeding pump users today. Users complain of the many alarms, beeps, and other operating noises, as they can significantly impact quality of life, sleep, and ability for the patient to take part in daily activities without feeling stigmatized or isolated. Additionally, when ambulating with a pump, the patient is often required to have a large backpack in which the pump and all other supplies must be placed, making it difficult for tube feeders to be discreet about their condition when in public. Further, the patient may be required to have multiple tubes and connector pieces to set up the enteral feeding pump. Additionally, the patient may also need multiple bag sets to be used in the operation of the enteral feeding pump.

Currently, enteral feeding patients operating an enteral feeding pump must connect a set of tubes to an enteral pump and connect connectors to the position where enteral feeding is to be delivered to the patient and the source of nutrition. Further, existing methods for administration of enteral nutrition often require nutritional fluid to be transferred from a nutrition source (e.g., carton) to a syringe or bag that can be coupled to a tube set for delivery of the feed to the feeding tube, thereby requiring more supplies and introducing potential sources of contamination to the nutritional fluid prior to delivery of the feed. Moreover, these existing methods are not very discrete, and may still cause a user or patient to feel stigmatized or isolated. In addition, these methods for administration of enteral nutrition often require the assistance of a caregiver, and can be cumbersome with the amount of supplies needed. Current options for continuous feeding via a peristaltic pump are not ideal for patients on-the-go, away from home or in a clinical setting.

Accordingly, improved systems for administration of an enteral feeding are desired in the art. In particular, a combination device which provides direct connection from a feed source to a patient's feeding tube without the need for any additional extension set or connections would be advantageous.

### SUMMARY

Objects and advantages of the invention will be set forth in part in the following description, or may be obvious from the description, or may be learned through practice of the invention.

In accordance with one aspect, an enteral feeding system is provided. The enteral feeding system includes a nutrition adapter including a coupling configured to couple to a source of enteral nutrition; a first tube having a proximal end and a distal end, and a lumen extending between the proximal end and the distal end. Specifically, the proximal end is coupled to the coupling of the nutrition adapter downstream of the coupling. Further, the enteral feeding system includes a cartridge configured to be inserted into a peristaltic pump. Specifically, the cartridge includes a cartridge tube defining a cartridge inlet to a cartridge outlet, and the distal end of the first tube is coupled to the cartridge inlet. Further, the enteral feeding system includes a second tube having a proximal end and a distal end, and a lumen extending between the proximal end and the distal end. Specifically, the proximal end is coupled to the cartridge outlet. Further, the enteral feeding system includes a feeding tube adapter coupled to the distal end of the second tube. Specifically, the feeding tube adapter includes a coupling configured to be coupled to a feeding tube receiving port, and the feeding tube adapter includes a lumen fluidly coupling the lumen of the second tube with a lumen of the feeding tube receiving port. Furthermore, the nutrition adapter, the first tube, the cartridge, the second tube, and the feeding tube adapter are formed as one piece.

In one embodiment of the enteral feeding system, the enteral feeding system further includes a solvent or adhesive bond between the proximal end of the first tube and the nutrition adapter.

In another embodiment of the enteral feeding system, the enteral feeding system further includes a solvent or adhesive bond between the distal end of the first tube and the cartridge inlet and a solvent or adhesive bond between the proximal end of the second tube and the cartridge outlet.

In a further embodiment of the enteral feeding system, the enteral feeding system further includes a solvent or adhesive bond between the distal end of the second tube and the feeding tube adapter.

In an additional embodiment of the enteral feeding system, the cartridge tube is formed from a first material, and at least one of the first tube and the second tube is formed from a second material, and the first material and the second material are different materials

In yet another embodiment of the enteral feeding system, the first material and the second material are each flexible materials.

In still another embodiment of the enteral feeding system, the feeding tube adapter coupling includes a male coupling configured to be received within a female coupling of the feeding tube receiving port, and wherein the male coupling includes at least one rib protruding from an external surface of the male coupling.

In yet a further embodiment of the enteral feeding system, an angle exists between a proximal end of the lumen of the feeding tube adapter and a distal end of the lumen of the feeding tube adapter in a range from about 70 degrees to about 110 degrees

In still a further embodiment of the enteral feeding system, the cartridge extends in a U-shape from the cartridge inlet to the cartridge outlet.

In another additional embodiment of the enteral feeding system, the cartridge includes at least one securing portion, the at least one securing portion formed along the U-shape of the cartridge tube.

In a further additional embodiment of the enteral feeding system, a continuous lumen is formed between the coupling of the nutrition adapter and the coupling of the feeding tube adapter.

In yet another additional embodiment of the enteral feeding system, the source of enteral nutrition includes an original source of nutrition, and the original source of nutrition is a single-use, disposable nutrition package.

In still another additional embodiment of the enteral feeding system, the nutrition adapter, the first tube, the cartridge, the second tube, and the feeding tube adapter are irremovably formed as one piece.

In another embodiment, the enteral feeding system includes an inline access port, where the nutrition adapter, the first tube, the cartridge, the second tube, the feeding tube adapter, and the inline access port are irremovably formed as one piece.

In accordance with another aspect, a kit for enteral feeding is provided. The kit includes a nutrition adapter including a coupling configured to couple to a source of enteral nutrition. The kit further includes a first tube having a proximal end and a distal end, and a lumen extending between the proximal end and the distal end where the distal end is coupled to the coupling of the nutrition adapter downstream of the coupling. The kit further includes a cartridge configured to be inserted into a peristaltic pump, including a cartridge tube extending in a U-shape from a cartridge inlet to a cartridge outlet where the proximal end of the first tube is coupled to the cartridge inlet. The kit further includes a second tube having a proximal end and a distal end, and a lumen extending between the proximal end and the distal end where the distal end is coupled to the cartridge outlet. The kit further includes a feeding tube adapter coupled to the proximal end of the second tube where the feeding tube adapter includes a coupling configured to be coupled to a feeding tube receiving port where the feeding tube adapter includes a lumen fluidly coupling the lumen of the second tube with a lumen of the receiving port. The kit further includes a support where the support is configured to retain the source of nutrition and the peristaltic pump in proximity to a patient. Furthermore, in the same aspect, the nutrition adapter, the first tube, the cartridge, the second tube, and the feeding tube adapter are formed as one piece.

In one embodiment, the support comprises a bag wearable by the patient.

In accordance with another aspect, a method for delivering enteral feed to a patient is provided. The method includes providing an enteral feeding system. The enteral feeding system includes a nutrition adapter including a coupling configured to couple to a source of enteral nutrition; a first tube having a proximal end and a distal end, and a lumen extending between the proximal end and the distal end. Specifically, the distal end is coupled to the coupling of the nutrition adapter downstream of the coupling. Further, the enteral feeding system includes a cartridge configured to be inserted into a peristaltic pump. Specifically, the cartridge includes a cartridge tube defining a cartridge inlet to a cartridge outlet, and the proximal end of the first tube is coupled to the cartridge inlet. Further, the enteral feeding system includes a second tube having a proximal end and a distal end, and a lumen extending between the proximal end and the distal end. Specifically, the distal is coupled to the cartridge outlet. Further, the enteral feeding system includes a feeding tube adapter coupled to the proximal end of the second tube. Specifically, the feeding tube adapter includes a coupling configured to be coupled to a feeding tube receiving port, and the feeding tube adapter includes a lumen fluidly coupling the lumen of the second tube with a lumen of the feeding tube receiving port. Furthermore, the nutrition adapter, the first tube, the cartridge, the second tube, and the feeding tube adapter are formed as one piece. The method further includes joining the coupling of the nutrition adapter to the source of enteral nutrition. The method further includes joining the coupling of the feeding tube adapter to the feeding tube receiving port. The method further includes arranging the cartridge within the peristaltic pump. The method further includes operating the peristaltic pump such that the enteral feed is delivered to the patient from the source of enteral nutrition through the enteral feeding system.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of an enteral feeding system according to one particular embodiment of the present disclosure;

FIG. 2 illustrates a perspective view of tube of an enteral feeding system according to one particular embodiment of the present disclosure;

FIG. 3 illustrates a perspective view of the enteral feeding system of FIG. 1, particularly showing the enteral feeding system being used with a peristaltic pump according to one particular embodiment of the present disclosure;

FIG. 4 illustrates a perspective view of an enteral feeding system being positioned for coupling to a source of enteral nutrition according to a particular embodiment of the present disclosure;

FIG. 5 illustrates a perspective view of an enteral feeding system being positioned for coupling to a feeding tube according to a particular embodiment of the present disclosure;

FIG. 6 illustrates a perspective view of another enteral feeding system being positioned for coupling to a feeding tube according to a particular embodiment of the present disclosure;

FIG. 7 illustrates a perspective view of an enteral feeding system according to the present disclosure, particularly illustrating an inline access port provided with the enteral feeding system;

FIG. 8 illustrates a perspective view of a support used with a kit for enteral feeding according to a particular embodiment of the present disclosure; and

FIG. 9 illustrates a flow chart of a method for delivering enteral feed to a patient in accordance with a particular embodiment of the present disclosure.

### DETAILED DESCRIPTION

Reference now will be made in detail to embodiments of the disclosure, one or more examples of which are illustrated in the drawings. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment can be used with another embodiment to yield a still further embodiment. Thus, it is intended that the present disclosure covers such modifications and variations as come within the scope of the appended claims and their equivalents. The detailed description uses numerical and letter designations to refer to features in the drawings. Like or similar designations in the drawings and description have been used to refer to like or similar parts of the disclosure.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Terms of approximation, such as “about,” “generally,” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise. Further, when a plurality of ranges are provided, any combination of a minimum value and a maximum value described in the plurality of ranges are contemplated by the present disclosure. For example, if ranges of “from about 20% to about 80%” and “from about 30% to about 70%” are described, a range of “from about 20% to about 70%” or a range of “from about 30% to about 80%” are also contemplated by the present disclosure.

As will be used herein, the terms proximal and distal each refer to a point of reference in relation to a particular object. Specifically, the term proximal end is defined as being situated nearer the object of reference, while the term distal is defined as being situated further from the object of reference. As described herein, the object of reference is the source of nutrition. Thus, an object that is proximal end is closer to the source of nutrition, while an object that is distal is further from the source of nutrition.

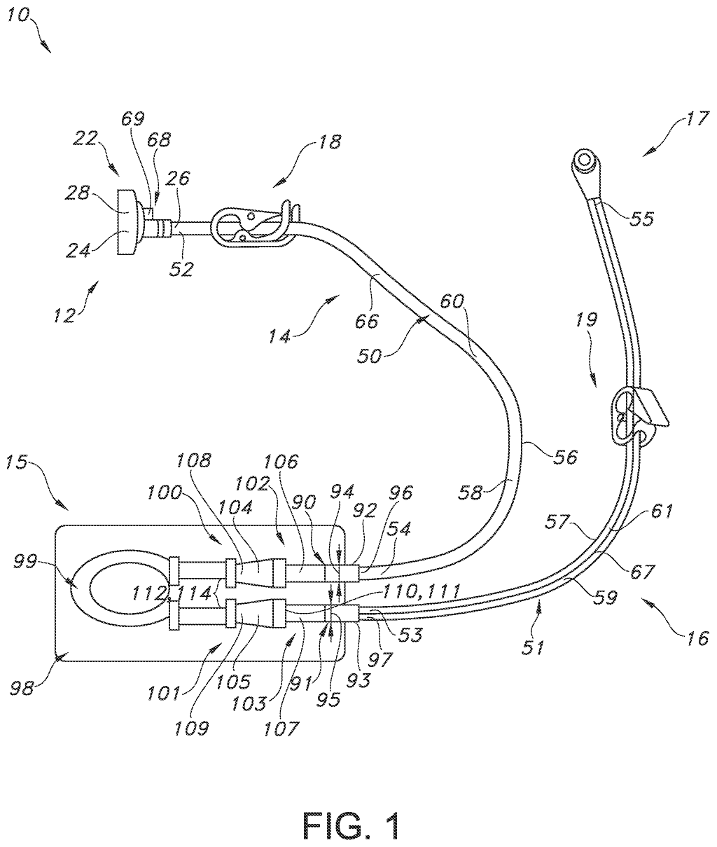

Generally speaking, the present disclosure is directed to an enteral feeding system having a nutrition source adapter, a first tube, a cartridge configured to be inserted into a peristaltic pump, a second tube, and a feeding tube adapter formed as a single integrated extension set. The enteral feeding system device includes a built-in adapter that connects directly to the container where the nutrition is stored on one end and connects directly to the feeding tube port on the other end. The device is also configured to be used with a peristaltic pump without any further components beyond the built-in adapter. Thus, the device is capable of providing an all-in-one giving set and extension set that removes the need to transfer nutrition from the nutrition source container to a syringe or a giving set bag for feeding. Additionally, the present disclosure is directed to a kit for enteral feeding that includes an enteral feeding system and a support, such as a garment or a bag, to hold a source of nutrition or a peristaltic pump to enable enteral feeding on-the-go. The specific features of the enteral delivery system and kit of the present disclosure may be better understood with reference to FIGS. 1-8.

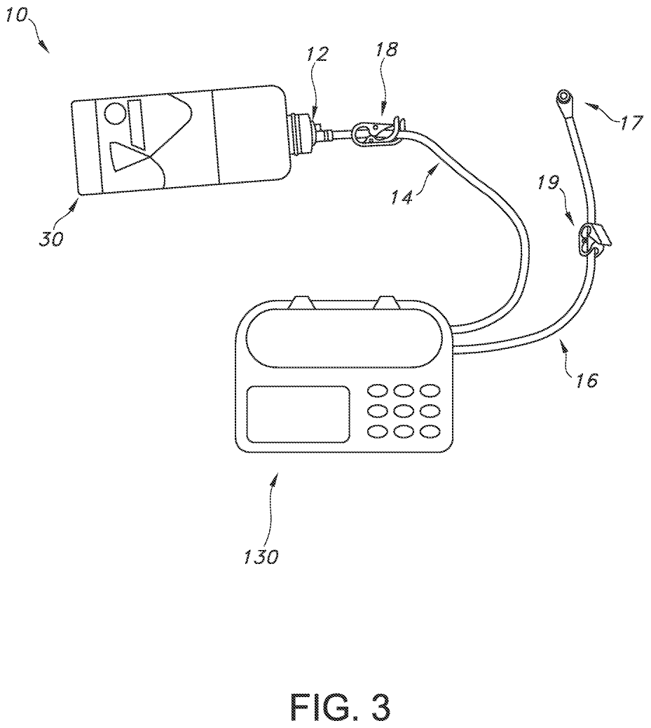

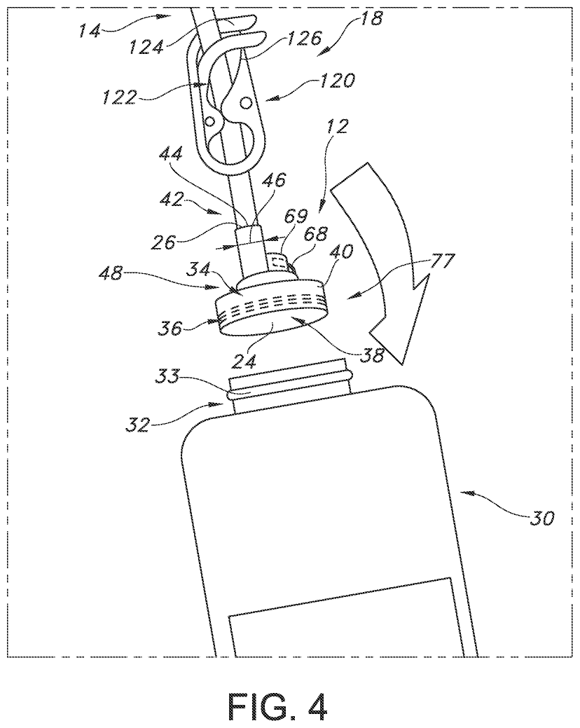

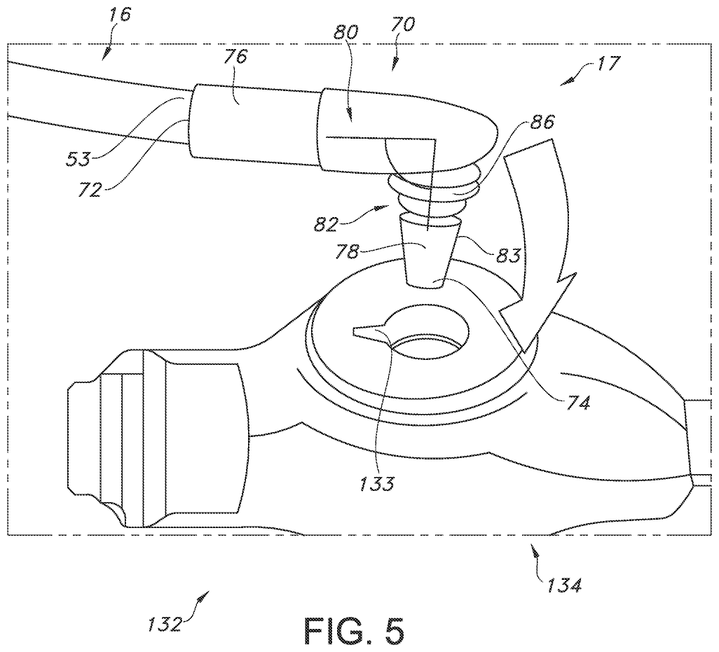

Referring now to FIG. 1, one embodiment of an enteral feeding system 10 is shown. The enteral feeding system 10 includes a nutrition adapter 12 configured to be coupled to a nutrition source (see FIG. 4), a first tube 14, a cartridge 15 configured to be inserted into a peristaltic pump (see FIG. 3), a second tube 16, and a feeding tube adapter 17 configured to be coupled directly with an indwelling feeding tube 132 (see FIG. 5). The enteral feeding system 10 may additionally include a flow restriction mechanism 18 configured to enable or occlude flow of nutrition through the first and second tubes 14, 16. For instance, the flow restriction mechanism 18 may be formed as a clamp.

The nutrition adapter 12 has a body 22 extending from a proximal end 24 to a distal end 26. The proximal end 24 includes a nutrition connector 28 configured and designed to complementarily attach directly to a connector 32 of source of nutrition 30 (see FIG. 4). The source of nutrition 30 may be a bag, a carton formed from plastic, cardboard or other suitable material, a reusable nutrition container (e.g., formed from plastic, thermoplastic or glass), or any other container for nutrition having a cooperating connector. In particular, the source of nutrition 30 to which the nutrition adapter 12 is designed to be coupled is intended to be an original source of nutrition, e.g., nutritional formula in a single-use package for bolus feeding as packaged by a manufacturer or source; however, reusable containers may also be coupled to the nutrition adapter 12. For instance, the proximal end 24 may include a collar 34 having threads 36 formed on an inner surface 38 or outer surface 40 of the collar 34. In other aspects of the present disclosure, the nutrition connector 28 may include a spike adapter (not shown) having a conical or cylindrical shape configured to be inserted into a source of nutrition 30. In still other aspects of the present disclosure, the nutrition connector 28 may include a push-on plug body configured for coupling to a plug receiver of a source of nutrition 30. The connector 32 of the source of nutrition 30 may include matching coupling features. For example, if the collar 34 has threads 36, the connector 32 may also include matching threads 33 that correspond with the threads 36.

Referring now to FIG. 4, at the distal end 26 of the nutrition adapter 12, the body 22 includes a tube coupling 42. The tube coupling 42 includes a cylindrical outer surface 44 having a diameter 46 configured to correspond to an inner diameter or outer diameter of the first tube 14, as described in further detail below. Thus, the tube coupling 42 may be inserted into the first tube 14 or the tube coupling 42 may surround an outer diameter of the first tube 14. In either instance, a distal end 54 of the first tube 14 is coupled to the nutritional adapter 12 downstream of the tube coupling 42. Further, the tube coupling 42 and the first tube 14 may be permanently, i.e., irremovably, coupled together. For instance, the tube coupling 42 and the first tube 14 may be solvent bonded, adhesively bonded, or coupled by any other suitable means such that the tube coupling 42 and the first tube 14 may not be separated without destroying the integrity of the first tube 14. As a result of this bonding, the tube coupling 42 and the first tube 14 may be formed as one piece when used in the enteral feeding system 10.

In any instance, the body 22 of the nutrition adapter 12 includes a nutrition lumen 48 extending therethrough from the nutrition connector 28 to the tube coupling 42. In this arrangement, when the nutrition adapter 12 is connected to a source of nutrition 30, the nutrition fluid can flow from the source of nutrition 30 through the length of the body 22 from the nutrition connector 28 through the tube coupling 42 and into the first tube 14.

The body 22 of the nutrition adapter 12 may be formed from any suitable thermoplastic material, including but not limited to acrylonitrile butadiene styrene (ABS), polycarbonate, polypropylene, and other materials. A suitable thermoplastic material for the body 22 is rigid and can be formed, e.g., by molding, into the shape of the body 22.

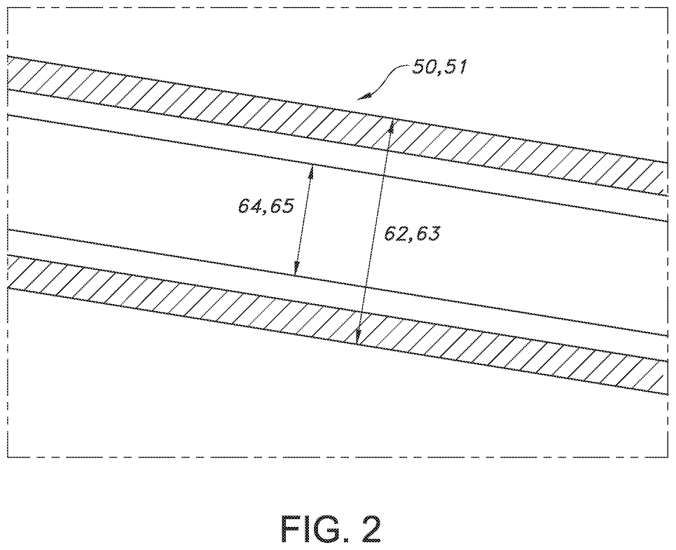

The first and second tubes 14, 16 each have a body 50, 51 that extends from a proximal end 52, 53 to a distal end 54, 55. Further, as shown in FIG. 2, the body 50 of the first and second tubes 14, 16 include an outer wall 56, 57 and an inner wall 58, 59. The inner walls 58, 59 may be concentrically disposed within the outer walls 56, 57. A lumen 60, 61 for each of the first and second tubes 14, 16 is defined by the inner walls 58, 59 and extends along the length of the bodies 50, 51 from the proximal ends 52, 53 to the distal ends 54, 55. The bodies 50, 51 have an outer diameter 62, 63 defined by the outer walls 56, 57 and an inner diameter 64, 65, also defined as a lumen diameter, defined by the inner walls 58, 59. The bodies 50, 51 of the first and second tubes 14, 16 may be formed from any suitable flexible material used for delivery of nutritional fluid, including but not limited to flexible polyvinyl chloride (PVC), silicone, polyurethane, and other thermoplastic elastomers.

A flow rate of nutrition through the first and second tubes 14, 16 can be determined at least in part by the inner diameters 64, 65 of the first and second tubes 14, 16 and a length 66, 67 extending from the proximal ends 52, 53 to the distal ends 54, 55 of the tubes 14, 16. In general, the larger the inner diameters 64, 65, the higher the flow rate of nutrition through the first and second tubes 14, 16. However, the flow rate of nutrition through the first and second tubes 14, 16 may be influenced by additional factors, including but not limited to the length and diameter of the nutrition lumen 48 through the body 22 of the nutrition adapter 12, the presence of a flow restriction mechanism 18, the viscosity and/or composition of the nutrition delivered through the first and second tubes 14, 16, and other factors.

The nutrition adapter 12 may also include multiple ports such that multiple sources of nutrition or other fluids may be connected to the first tube 14. For example, referring to FIG. 4, the nutrition adapter 12 may include a first inlet port 77 configured to couple with the source of enteral nutrition 30 and a second inlet port 68 configured to couple with a secondary fluid source (not shown). The secondary fluid source may be another source of enteral nutrition 30, or alternatively, the secondary fluid source may be a medicine such that feeding and medicating are accomplished with the same enteral feeding system 10. In addition, the nutrition adapter 12 may further include a tethered cap 69 configured to seal the second inlet port 68. By providing the tethered cap 69, second inlet port 68 may be kept sterile until it is connected to the secondary fluid source.

Between the first and second tubes 14, 16, the cartridge 15 is attached. Specifically, the cartridge 15 is located between the proximal end 52 of the first tube 14 and the distal end 55 of the second tube 16. The cartridge 15 includes tube couplings 90, 91. Like tube coupling 42, tube couplings 90, 91 each include a cylindrical outer surface 92, 93 having a diameter 94, 95 configured to correspond to an inner diameter or outer diameter of the first and second tubes 14, 16. In addition, the tube couplings 90, 91 may be inserted into the first and second tubes 14, 16, or the tube couplings 90, 91 may surround an outer diameter of the first and second tubes 14, 16. In either instance, one end of the first tube 14 and one end of the second tube 16 are coupled to the cartridge 15. Further, the tube couplings 90, 91 and the first and second tubes 14, 16 may be permanently, i.e., irremovably, coupled together. For instance, the tube couplings 90, 91 and the first and second tubes 14, 16 may be solvent bonded, adhesively bonded, or coupled by any other suitable means such that the tube couplings 90, 91 and the first tube 14 may not be separated without destroying the integrity of the first and second tubes 14, 16. Consequently, like the tube coupling 42 and the first tube 14, the tube couplings 90, 91 and the first and second tubes 14, 16 may be formed as one piece when used in the enteral feeding system 10.

Referring now to FIG. 3, the cartridge 15 is configured to be inserted into a peristaltic pump 130. For example, the shape of the frame 98 of the cartridge 15 may be customized to fit within any known peristaltic pump used for enteral feeding. The cartridge 15 includes a cartridge tube 99 which can extend in a U-shape from a cartridge inlet 100 to a cartridge outlet 101, The cartridge 15 may be formed of particularly advantageous materials. For example, the cartridge tube 99 may be formed from materials similar to the first and second tubes 14, 16 including, but not limited to, flexible polyvinyl chloride (PVC), silicone, polyurethane, and other thermoplastic elastomers. The cartridge tube 99 may also be formed from materials differing from the first and second tubes 14, 16. For example, the first and second tubes 14, 16 may be formed form a PVC while the cartridge tube 99 is formed from a silicone based elastomer. By forming the cartridge tube 99 from [type of material], the peristaltic pump 130 may be able to exert force better on the cartridge 15 thereby improving the overall flow rate of bolus feed through the enteral feeding system 10.

The other portions of the cartridge 15, such as the frame 98 and the cartridge inlet/outlet 100, 101, may be formed from another type of material. For example the frame 98 and the cartridge inlet/outlet 100, 101 may be formed from a rigid material including but not limited to any suitable type of thermoplastic.

The cartridge inlet 100 and the cartridge outlet 101 are configured for coupling to the first and second tubes 14, 16 at the tube couplings 90, 91. Specifically, the cartridge inlet 100 and the cartridge outlet 101 can include the tube couplings 90, 91, respectively. The tube couplings 90, 91 can be connected to the first and second tubes 14, 16 as aforementioned. Further, the cartridge inlet 100 and the cartridge outlet 101 can include connecting portions 102, 103 between the tube couplings 90, 91 and the cartridge tube 99. The connecting portions 102, 103 can include tapered portions 104, 105 and longitudinal portions 106, 107. The tapered portions 104, 105 may have a similar diameter as the cartridge tube 99 at first ends 108, 109 and a wider, expanded diameter at second ends 110, 111. The longitudinal portions 106, 107 may have a diameter approximately the same as the tube couplings 90, 91 such that the tube couplings 90, 91 fit around or fit within the longitudinal portions 106, 107. The cartridge 15 may also include securing portions 112, 114. The securing portions 112, 114 may be configured to secure the cartridge tube 99 into a U-shape. Particularly, the securing portions 112, 114 may be placed such that the cartridge tube 99 is bent into a U-shape when inserted into the securing portions 112, 114.

Referring now to FIG. 5, a feeding tube adapter 17 is coupled to the proximal end 53 of the second tube 16. The feeding tube adapter 17 has a body 70 that extends from a proximal end 72 to a distal end 74. The body 70 is coupled to the proximal end 53 of the second tube 16 at the distal end 74. For instance, the distal end 74 may include a collar 76 configured to be inserted within the lumen 61 of the second tube 16 and/or configured to receive the outer wall 56 of the second tube 16 therein within the collar 76. Similarly to the connection between the first tube 14 and the nutrition adapter 12 at the distal end 54 of the first tube 16, the body 70 of the feeding tube adapter 17 and the second tube 16 may be permanently, i.e., irremovably, coupled together. For instance, the collar 76 and the second tube 16 may be solvent bonded, adhesively bonded, or coupled by any other suitable means such that the body 70 and the second tube 16 may not be separated without destroying the integrity of the second tube 16. As a result, the body 70 and the second tube 16 may be one piece when used in the enteral feeding system 10.

Further, the enteral feeding system 10 is formed as a single unit. Stated differently, the nutrition source adapter 12, the first tube 14, the cartridge 15, the second tube 16, and the feeding tube adapter 17 are irremovably coupled together such that none of the components can be separated from each other without destroying the system 10 and rendering the system 10 inoperable and unsuited for its intended purpose of the delivery of a bolus feed to a feeding tube 132. For example, the continuous feed delivery system 10 can be welded together using a technique such as ultrasonic welding or thermal welding. Consequently, the nutrition source adapter 12, the first tube 14, the cartridge 15, the second tube 16, and the feeding tube adapter 17 may be formed together as one piece when used in the enteral feeding system 10.

At the proximal end 72 of the body 70, the feeding tube adapter 17 includes a feeding tube coupling 78. The feeding tube coupling 78 is configured to be inserted directly into a receiving port 134 (see FIG. 5) of an indwelling feeding tube 132 having a lumen. A feeding tube adapter lumen 80 extends through the body 70 from the distal end 74 to the proximal end 72 to fluidly couple the feeding tube adapter 17 with the second tube 16 such that nutrition may be delivered from the second tube 16 to the feeding tube 132 when the coupling 78 is inserted within the receiving port 134 of the feeding tube 132 (see FIG. 5).

The feeding tube coupling 78 may comprise a stem 82 configured to be inserted into the receiving port 134 of the indwelling feeding tube 132. The stem 82 may form a male connector or coupling configured to be inserted into a female receiving coupling or port 134 of the indwelling feeding tube 132. The stem 82 may have a cylindrical cross-sectional shape or any other rounded cross-sectional shape. The stem 82 may have a tapered proximal end having a diameter that tapers toward the terminal proximal end 72 of the stem 82.

The stem 82 and proximal end 72 of the body 70 may extend at an angle with respect to the distal end 74. For instance, the stem 82 and proximal end 72 of the body 70 may extend from an axis parallel to the body 70 at an angle in a range from about 70 degrees to about 110 degrees with respect to the distal end 74. More particularly, the proximal end 72 and distal end 74 may be generally perpendicular to each other, in other words, at about a 90 degree angle relative to each other, as shown in FIG. 5. The present inventors have found that providing an angled configuration, such as a generally perpendicular angle, between the proximal end 72 and distal end 74 of the body 70 can form a low-profile feeding tube adapter 17 that may be easily concealed beneath a garment worn by a feeding tube 132 for a patient (see FIG. 5). Further, the present inventors have found that providing a feeding tube adapter 17 having a bent configuration provides a prolonged connection for a continuous delivery of nutrition to the patient resulting in a slowly deliver of a particular volume of nutritional fluid to the patient over a period of time. Such a configuration may be suitable for enteral feeding using the enteral feeding system 10.

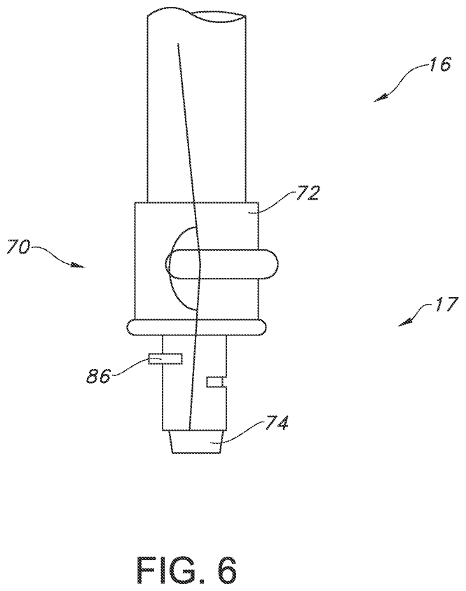

In another aspect of the present disclosure, as illustrated in FIG. 6, the body 70 of the feeding tube adapter 17 can extend in a continuous longitudinal or linear direction such that there is a generally straight line extending between the proximal end 72 and the distal end 74 of the body 70. Stated differently, a generally straight line may extend from the proximal end 72 through the stem 82. For instance, the stem 82 can extend along an axis parallel with the body 70 at an angle of from about 165 degrees to about 195 degrees, such as from about 170 degrees to about 190 degrees, for instance from about 175 degrees to about 185 degrees, for instance at 180 degrees, with respect to the distal end 74 of the body 70. In one aspect of the present disclosure, the proximal end 72 of the lumen 80 may extend at an angle of about 180 degrees from the distal end 74 of the lumen 80, i.e., the lumen 80 extends in a generally straight line.

As shown in FIG. 5, the body 70 may include one or more mating features 86 configured to removably couple with complementary mating or locking features 133 of the receiving port 134 of the feeding tube 132. For example, the body 70 may include one or more ribs or protrusions configured to be received within a complementary receiving port 134 of the feeding tube 132. For example, the ribs may protrude from an external surface 83 of the stem 82. As stated hereinabove, the receiving port 134 may also include mating features 86. For example, the mating features may include a groove that is sized for the insertion of either the stem 82 or the mating features 86 of the body 70.

As shown in FIGS. 1 and 4, a flow restriction mechanism 18 may be coupled to the first or second tubes 14, 16 to restrict or control the flow of fluid from the nutrition source through the first or second tubes 14, 16. For instance, the flow restriction mechanism 18 may be formed as a clamp 120 as shown in FIG. 4. The clamp 120 may include at least one aperture 122 through which the tube body 50 extends. Further, the clamp 120 may include at least one restriction member 124 configured to be positioned in an open or closed configuration. In the open configuration, fluid may flow through the first and second tubes 14, 16 without restriction from the flow restriction mechanism 18. In the closed configuration, the restriction member 124 may be compressed against the tube body 50 and held in place by a locking member 126. The restriction member 124 may thereby compress the tube body 50 to prevent and/or restrict flow of fluid through the lumens 60, 61 at the point at which the tube body 50 is compressed. In some aspects, the restriction member 124 may full prevent flow of fluid through the lumens 60, 61. In other aspects of the present disclosure, the restriction member 124 may restrict or limit, i.e., slow, the flow of fluid through the lumens 60, 61 to decrease the flow rate of fluid through the first and second tubes 14, 16.

In addition to or as an alternative to the push-style clamp 120, the flow restriction mechanism 18 may incorporate a roller-style clamp. A roller-style clamp may enable varying degrees of adjustment in the amount the tubing is compressed, and thus, modulate fluid flow through the tube. Still further, a rotary style flow restriction mechanism that inserts connectors of varying sizes inline to the tubing may be provided to modulate fluid flow through the first and second tubes 14, 16. These connectors could be formed from a variety of designs including, but not limited to, tubing segments and pieces of rigid plastic. A variety of lengths and sizes, e.g. diameter, of connectors for a rotary style flow restrictor could be offered to achieve different amounts of flow. In such an arrangement, a user could rotate a dial (or other mechanism) that adjusts the flow path of the fluid through one of the connectors, and thus, change the flow rate of fluid through the device.

More than one flow restriction mechanism 18 may also be provided. Specifically, a first flow restriction mechanism 18 may be provided on the first tube 14, while a second flow restriction mechanism 19 is provided on the second tube 16. Further, the first flow restriction mechanism 18 on the first tube 14 may differ from the second restriction mechanism 18 on the second tube 16. For example, the first flow restriction mechanism 18 on the first tube 14 may be provided to control the flow of a bolus feed through the first tube 14, but not entirely cease it, while the second flow restriction mechanism 19 on the second tube 16 may be provided to either allow the flow of a bolus feed or entirely halt the flow. By providing flow restriction mechanisms 18, 19 on both the first and second tubes 14, 16, the flow of enteral nutrition through the enteral feeding system 10 may be controlled before the enteral nutrition enters the cartridge 15 and after the enteral nutrition exits the cartridge 15. This allows for the flow of the enteral nutrition to be controlled both before and after the peristaltic pump 130 applies pressure to the enteral feeding system 10.

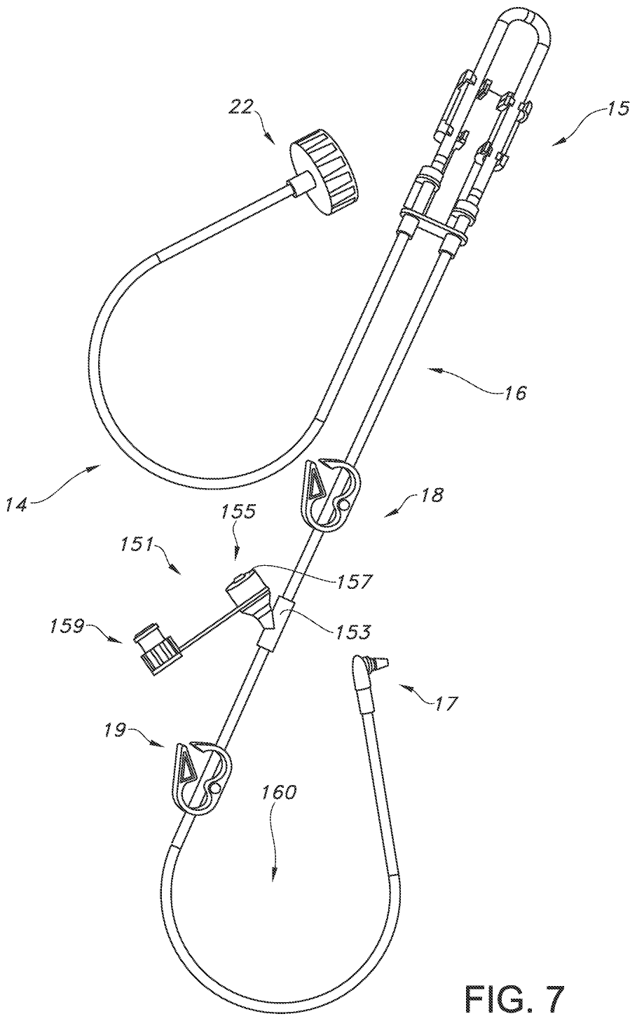

Referring now to FIG. 7, an embodiment of an enteral feeding system 10 is presented, particularly illustrating an inline access port 151 provided with the enteral feeding system 10. As shown, the inline access port 151 may be attached to the second tube 16 downstream of the nutrition source adapter 12 and the cartridge 15. Specifically, the inline access port 151 may include a tubular portion 153 that the second tube 16 is inserted into or attached with. The inline access port 151 may also include a branched portion 155, such as a Y-shaped portion, that allows for the provision of a second source of fluid such as nutrition or medicine. The inline access port 151 may also allow for the flushing of the second tube 16 if any nutrition or other matter remains in the second tube 16. The inline access port 151 may also allow for the decompressing or venting of the patient's stomach that is using the enteral feeding system 10. To achieve these features, the branched portion 155 may include a secondary connector 157. The secondary connector 157 may have a variety of shapes and configurations to attach to whatever the second source of fluid is provided. For example, the secondary connector 157 may be a threaded or screw-on connection or a press-fit connection.

In addition, the inline access port 151 may be solvent bonded, adhesively bonded, or coupled by any other suitable means to the second tube 16 such that the inline access port 151 is inseparable from the second tube 16. Further, the nutrition source adapter 12, the first tube 14, the cartridge 15, the second tube 16, the feeding tube adapter 17, and the inline access port 151 may be irremovably coupled together such that none of the components are separated from each other without destroying the system 10 and rendering the system 10 inoperable and unsuited for its intended purpose of the delivery of a bolus feed to a feeding tube. Consequently, the nutrition source adapter 12, the first tube 14, the cartridge 15, the second tube 16, the feeding tube adapter 17, and the inline access port 151 may all be formed as one piece when used in the enteral feeding system 10.

In another embodiment, a third tube 160 may be provided that connects between the inline access port 151 and the feeding tube adapter 17. If a third tube 160 is provided, the nutrition source adapter 12, the first tube 14, the cartridge 15, the second tube 16, the feeding tube adapter 17, the inline access port 151, and the third tube 160 may all be formed as one piece when used in the enteral feeding system 10 as described above.

In addition, as shown, the inline access port 151 may include a tethered cap 159. The tethered cap 159 may be attached to the branched portion 155 of the inline access port 151. Further, the tethered cap 159 may allow for the sealing of the inline access port 151, if it is not currently being used.

In addition, as shown, if the inline access port 151 is provided, the first or second flow restriction mechanisms 18, 19 may be provided downstream of both the nutrition adapter 12 and the inline access port 151. By placing the flow restriction mechanisms 18, 19 downstream, the flow restriction mechanisms 18, 19 may restrict or prevent the flow of nutrition from the nutrition adapter 12 or the inline access port 151.

A kit 200 for delivery of enteral nutrition via enteral feeding may further be provided. The kit 200 can include the enteral feeding system 10 described above and a support 202 configured to support, e.g., hold in place, a nutrition source 30. The nutrition source 30 may further be included in the kit 200. The support 202 can be in the form of a bag or garment to be worn by a patient or any other suitable support configured to hold the peristaltic pump 130 and engage with the nutrition source 30. For instance, an intravenous (IV) pole or other mount for supporting the nutrition source 30 or the peristaltic pump 130 may be provided as a support 202.

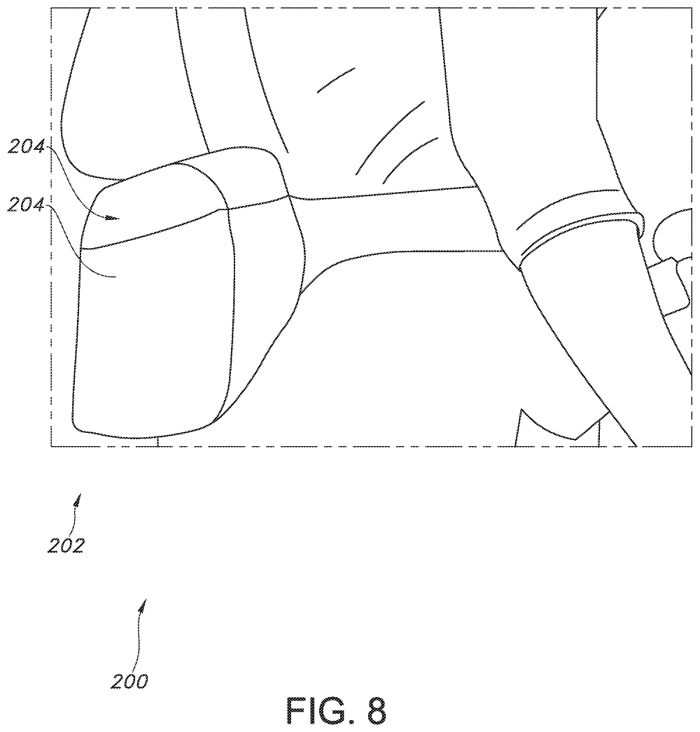

As shown in FIG. 8, the support 202 may include a bag 204 to be worn by a patient. The bag 204 can include a belt, a vest, or a harness having one or more straps to secure the bag 204 to the patient. The bag 204 may include one or more pockets 206 configured to hold or contain the nutrition source 30 and the peristaltic pump 130. The pocket(s) 206 can be disposed at any suitable portion of the bag 204 to enable the nutritional fluid to flow through the first and second tubes 14, 16 to the feeding tube 132. For example, a pocket 206 may be placed at the top of the bag 204 as shown. The bag 204 may be worn either over or concealed beneath a patient's clothing. By including a support 202 such as a wearable bag 204 to hold the nutrition source 30 and the peristaltic pump 130, the kit 200 may advantageously enable a patient to conduct a feeding on-the-go, i.e., away from home or a clinical setting.

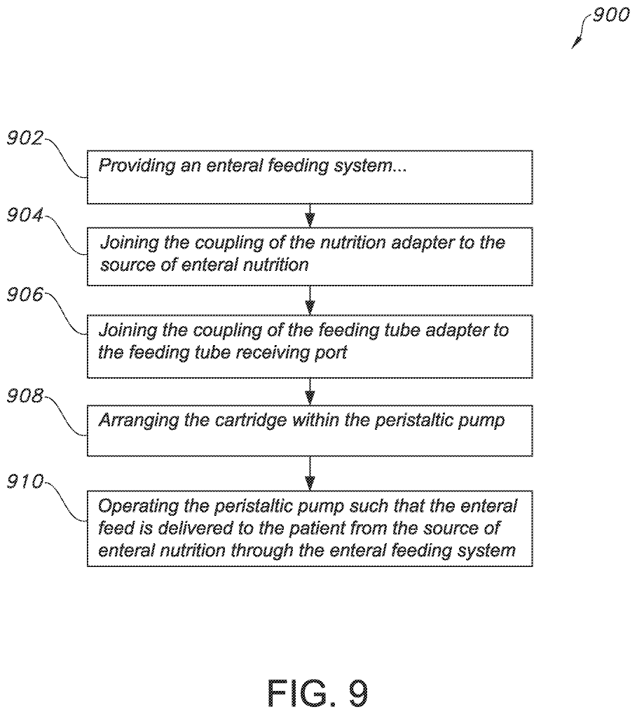

As shown in FIG. 9, the present disclosure may be further directed to a method for a continuous delivering of feed to a patient. The method 900 may be executed using the enteral feeding system 10 as detailed above. At step 902, the method includes providing an enteral feeding system, such as enteral feeding system 10. Next, at step 904, the method includes coupling the coupling of the nutrition adapter to the source of enteral nutrition. Next, at step 906, the method includes coupling the coupling of the feeding tube adapter to the feeding tube receiving port. Next, at step 908, the method includes arranging the cartridge within the peristaltic pump. Then, at step 910, the method includes operating the peristaltic pump such that the enteral feed is delivered to the patient from the source of enteral nutrition through the enteral feeding system.

An exemplary technical effect of the methods, systems, and apparatus described herein includes at least one of, but are not limited to: a) removing the need to transfer enteral nutrition from its original source to a separate container for enteral feeding, b) improving efficiency of delivery of a bolus feed, especially in an outpatient setting, and c) improving ease of preparation of an enteral feed for enteral feeding.

This written description uses examples to disclose the disclosure, including the best mode, and also to enable any person skilled in the art to practice the disclosure, including making and using any devices or systems and performing any incorporated methods. The patentable scope of the disclosure is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. An enteral feeding system comprising:
a nutrition adapter comprising a coupling configured to couple to a source of enteral nutrition;
a first tube having a proximal end and a distal end, and a lumen extending between the proximal end and the distal end, wherein the proximal end is coupled to the coupling of the nutrition adapter downstream of the coupling;
a cartridge configured to be inserted into a peristaltic pump, wherein the cartridge comprises a cartridge tube defining a cartridge inlet to a cartridge outlet, wherein the distal end of the first tube is coupled to the cartridge inlet;
a second tube having a proximal end and a distal end, and a lumen extending between the proximal end and the distal end, wherein the proximal end is coupled to the cartridge outlet; and
a feeding tube adapter coupled to the distal end of the second tube, wherein the feeding tube adapter comprises a coupling configured to be coupled to a feeding tube receiving port, wherein the feeding tube adapter comprises a lumen fluidly coupling the lumen of the second tube with a lumen of the feeding tube receiving port,
wherein the nutrition adapter, the first tube, the cartridge, the second tube, and the feeding tube adapter are formed as one piece.

2. The enteral feeding system of claim 1, further comprising a solvent or adhesive bond between the proximal end of the first tube and the nutrition adapter.

3. The enteral feeding system of claim 1, further comprising a solvent or adhesive bond between the distal end of the first tube and the cartridge inlet and a solvent or adhesive bond between the proximal end of the second tube and the cartridge outlet.

4. The enteral feeding system of claim 1, further comprising a solvent or adhesive bond between the distal end of the second tube and the feeding tube adapter.

5. The enteral feeding system of claim 1, wherein the cartridge tube is formed from a first material, wherein at least one of the first tube and the second tube is formed from a second material, and wherein the first material and the second material are different materials.

6. The enteral feeding system of claim 5, wherein the first material and the second material are each flexible materials.

7. The enteral feeding system of claim 1, wherein the feeding tube adapter coupling comprises a male coupling configured to be received within a female coupling of the feeding tube receiving port, wherein the male coupling comprises at least one rib protruding from an external surface of the male coupling.

8. The enteral feeding system of claim 1, wherein an angle between a proximal end of the lumen of the feeding tube adapter and a distal end of the lumen of the feeding tube adapter is in a range from about 70 degrees to about 110 degrees.

9. The enteral feeding system of claim 1, wherein the cartridge extends in a U-shape from the cartridge inlet to the cartridge outlet, wherein the cartridge comprises at least one securing portion, the at least one securing portion formed along the U-shape of the cartridge tube.

10. The enteral feeding system of claim 1, the enteral feeding system further comprises an inline access port, wherein the nutrition adapter, the first tube, the cartridge, the second tube, the feeding tube adapter, and the inline access port are irremovably formed as one piece.

11. The enteral feeding system of claim 1, wherein a continuous lumen is formed between the coupling of the nutrition adapter and the coupling of the feeding tube adapter.

12. The enteral feeding system of claim 1, wherein the source of enteral nutrition comprises an original source of nutrition, wherein the original source of nutrition is a single-use, disposable nutrition package.

13. The enteral feeding system of claim 1, wherein the nutrition adapter, the first tube, the cartridge, the second tube, and the feeding tube adapter are irremovably formed as one piece.

14. A kit for enteral feeding comprising:
a nutrition adapter comprising a coupling configured to couple to a source of enteral nutrition;
a first tube having a proximal end and a distal end, and a lumen extending between the proximal end and the distal end, wherein the proximal end is coupled to the coupling of the nutrition adapter downstream of the coupling;
a cartridge configured to be inserted into a peristaltic pump, comprising a cartridge tube extending in a U-shape from a cartridge inlet to a cartridge outlet, wherein the distal end of the first tube is coupled to the cartridge inlet;
a second tube having a proximal end and a distal end, and a lumen extending between the proximal end and the distal end, wherein the proximal end is coupled to the cartridge outlet;
a feeding tube adapter coupled to the distal end of the second tube, wherein the feeding tube adapter comprises a coupling configured to be coupled to a feeding tube receiving port, wherein the feeding tube adapter comprises a lumen fluidly coupling the lumen of the second tube with a lumen of the receiving port; and
a support, wherein the support is configured to retain the source of nutrition and the peristaltic pump in proximity to a patient,
wherein the nutrition adapter, the first tube, the cartridge, the second tube, and the feeding tube adapter are formed as one piece.

15. The kit of claim 14, wherein the support comprises a bag wearable by the patient.

16. The kit of claim 14, further comprising a solvent or adhesive bond between the distal end of the first tube and the cartridge inlet and a solvent or adhesive bond between the proximal end of the second tube and the cartridge outlet.

17. The kit of claim 14, wherein the cartridge tube is formed from a first material, at least one of the first tube and the second tube is formed from a second material, wherein the first material and the second material are different materials.

18. The kit of claim 17, wherein the first material and the second material are each flexible materials.

19. The kit of claim 14, wherein an angle between a proximal end of the lumen of the feeding tube adapter and a distal end of the lumen of the feeding tube adapter is in a range from about 70 degrees to about 110 degrees.

20. A method for delivering enteral feed to a patient, the method comprising:
providing an enteral feeding system comprising:
a nutrition adapter comprising a coupling configured to couple to a source of enteral nutrition,
a first tube having a proximal end and a distal end, and a lumen extending between the proximal end and the distal end, wherein the proximal end is coupled to the coupling of the nutrition adapter downstream of the coupling,
a cartridge configured to be inserted into a peristaltic pump, comprising a cartridge tube extending in a U-shape from a cartridge inlet to a cartridge outlet, wherein the distal end of the first tube is coupled to the cartridge inlet,
a second tube having a proximal end and a distal end, and a lumen extending between the proximal end and the distal end, wherein the proximal end is coupled to the cartridge outlet, and
a feeding tube adapter coupled to the proximal end of the second tube, wherein the feeding tube adapter comprises a coupling configured to be coupled to a feeding tube receiving port, wherein the feeding tube adapter comprises a lumen fluidly coupling the lumen of the second tube with a lumen of the receiving port, wherein the nutrition adapter, the first tube, the cartridge, the second tube, and the feeding tube adapter are formed as one piece;
joining the coupling of the nutrition adapter to the source of enteral nutrition;
joining the coupling of the feeding tube adapter to the feeding tube receiving port;
arranging the cartridge within the peristaltic pump; and
operating the peristaltic pump such that the enteral feed is delivered to the patient from the source of enteral nutrition through the enteral feeding system.
